(19) United States
(12) Patent Application Publication
(10) Pub. No.: US 2026/0285534 A1
(43) Pub. Date: Sep. 24, 2026

(54) SURGICAL DEVICE TRAY HAVING A HANDLE

(71) Applicant: Cilag GmbH International, Zug (CH)

(72) Inventors: Brandon L. Burton, Cincinnati, OH (US); Susan A. L. Diamond, Fort Wayne, IN (US); Clifford J. Labbe, JR., Cincinnati, OH (US); Steven M. Gleason, Cincinnati, OH (US); Stephen M. Christopher, Philadelphia, PA (US)

(73) Assignee: Cilag GmbH International, Zug (CH)

(21) Appl. No.: 19/538,466

(22) Filed: Feb. 12, 2026

Related U.S. Application Data
(60) Provisional application No. 63/774,884, filed on Mar. 20, 2025.

(51) Int. Cl. B65D 1/36; A61B 5/15; A61B 17/00; A61B 17/068; A61B 34/20; B65D 1/34; B65D 25/28

(52) U.S. Cl. CPC B65D 1/36; A61B 5/15003; A61B 17/068; A61B 34/20; B65D 1/34; B65D 25/2808; A61B 2017/00398; A61B 2017/00477

(57) ABSTRACT

Packaging for medical devices is disclosed. The packaging may include a tray having sidewalls that extend between a base and a rim to form a receptacle of varying depth. The sidewalls may include handle sidewalls extending to the base at the peak depth of the receptacle and forming a tray handle. The handle sidewalls may be laterally spaced to accommodate a handle of a surgical device and sized to deflect inwardly toward opposing lateral sides of the handle of the surgical device when manually grasped. The handle sidewalls may be thinner and more flexible than the other sidewalls of the tray. A surgical device may be stored in the tray in an upright orientation. The tray may be thermoformed.

### BACKGROUND

The present disclosure generally relates to medical devices and packaging and, more particularly, to packaging for medical devices.

Medical devices may be packaged for transportation and/or storage. The medical device packaging may be opened prior to or during a surgical procedure for removal of the medical device from the packaging. The medical device may be a sterilized surgical device. At least one member of an operating team may be responsible for opening the medical package and/or presenting the medical device therein to a sterile nurse or a surgeon while preserving the sterile condition of the medical device. Contact between a medical device and portions of the packaging for the medical device may defeat the sterile condition of the medical device.

Medical devices may be sealed and sterilized within the package. Packaging may include a peelable package for containing the medical device. Exemplary packaging is disclosed in U.S. Pat. No. 3,724,651 to Link, titled PEELABLE SURGICAL PACKAGE, U.S. Patent Application Publication No. 2009/0205988 to Edwards et al., titled ERGONOMIC DISPOSABLE TRAY FOR HOLDING STERILE SURGICAL COMPONENTS, and U.S. Patent Application Publication No. 2012/0061262 to Merboth et al., titled PACKAGES FOR MEDICAL DEVICES AND METHODS THEREFOR. For surgical devices comprising a handle and an elongate shaft, the surgical device is generally laid on its side in a packaging tray, as disclosed in U.S. Pat. No. 5,353,929 to Foster, titled PACKAGE FOR SURGICAL INSTRUMENT and U.S. Pat. No. 9,265,578 to Dacey, titled MULTI-COMPONENT PACKAGES FOR MEDICAL DEVICES, and U.S. Patent Application Publication No. 2003/0121821 to Roshdy, titled BLISTER TRAY WITH A PACKAGE FOR A SMALL DEVICE, for example.

### SUMMARY

One general aspect includes a container. The container may include a tray having a base, a rim, and a plurality of sidewalls extending between the base and the rim to form a receptacle that has a varying depth. The plurality of sidewalls may include a plurality of handle sidewalls extending from the rim to the base at a peak depth along the varying depth of the receptacle. A surgical device may be positioned in the receptacle; the surgical device may include a handle and a shaft extending from the handle along a shaft axis, where the handle may include a grip extending along a grip axis oriented transverse to the shaft axis. The grip axis may extend through the base of the tray to orient the handle in an upright orientation in the receptacle.

Implementations of the container may include one or more of the following features. The peak depth may be greater than the width and less than the length of the receptacle. The handle may further include a height, and the peak depth may be greater than the height. The grip may include a pair of opposing lateral sides, and the plurality of handle sidewalls may be adjacent to the pair of opposing lateral sides. The surgical device may be a sterile surgical device. The surgical device further may include an end effector extending distally from the shaft, and the base may further include a crest positioned to support the shaft at a location intermediate the handle and the end effector. The receptacle may further include a distal cavity, and the end effector may be suspended in the distal cavity of the receptacle. The surgical device may further include a coiled cord positioned in the receptacle, and the coiled cord may be oriented in an upright orientation substantially parallel to the grip axis of the surgical device.

One general aspect includes a container including a tray having a base, a rim, and a plurality of sidewalls extending between the base and the rim, where the base and the plurality of sidewalls form a receptacle, and where the receptacle may include: a first end, a second end opposite to the first end, and a varying depth including a peak depth intermediate the first end and the second end. The plurality of sidewalls may include a plurality of handle sidewalls extending to the base at the peak depth of the receptacle, and the plurality of handle sidewalls may form a tray handle. The plurality of handle sidewalls may be laterally spaced to accommodate a handle of a surgical device and sized to deflect inwardly toward opposing lateral sides of the handle of the surgical device when manually grasped.

Implementations of the container may include one or more of the following features. The plurality of sidewalls may further include a distal pair of sidewalls extending from the base to the rim, where the distal pair of sidewalls are distal to the plurality of handle sidewalls, and where a minimum wall thickness of the distal pair of sidewalls is greater than the minimum wall thickness of the handle sidewalls. The plurality of handle sidewalls may be more flexible than the distal pair of sidewalls. The plurality of handle sidewalls may include an outer surface having a textured grip. The rim may define an opening into the receptacle, and the opening may define an opening surface area, where the receptacle defines a volume, and where the ratio of the volume to the opening surface area is greater than 1.5 inches, such as 1.65 inches, for example. The rim may define an opening into the receptacle, where the opening defines a perimeter length, where the receptacle defines a volume, and where the ratio of the volume to the perimeter length is greater than 3 inches, such as 3.1 inches, for example. The container may include a flexible film adhered to the rim along the perimeter length. The surgical device may be positioned in an upright orientation in the receptacle, and the surgical device may be encased within the receptacle between the tray and the flexible film. The tray insert may include a plurality of contours structured to complement an upper surface of the surgical device positioned in the upright orientation in the receptacle.

One general aspect includes a set of surgical device packaging containers including a pair of identical trays each including a base and a plurality of sidewalls extending from the base to form a receptacle. The bases may each include an outward-facing surface including a protrusion and an indentation. The outward-facing surfaces of the identical containers may face each other such that the protrusions are configured to nest in the complementary indentations of the other tray.

Implementations of the set of surgical device packaging containers may include one or more of the following features. The set may include a first surgical device positioned in a first tray, and a second surgical device positioned in a second tray. The first surgical device may include a handle, a shaft, and an end effector, where the protrusion of the first tray is dimensioned to receive the handle and the indentation of the first tray is structured to support the shaft at a location between the handle and the end effector. The first and second surgical devices may be identical or non-identical.

One general aspect includes a method. The method may include obtaining a sterile surgical device including a handle and a shaft extending from the handle along a shaft axis, where the handle includes a grip, and where the grip extends along a grip axis oriented transverse to the shaft axis. The method may further include obtaining a tray having a base and a plurality of sidewalls extending from the base to form a receptacle, where the plurality of sidewalls may include a proximal sidewall and a distal sidewall. The method may further include positioning the sterile surgical device in the tray in an upright orientation, where, in the upright orientation, the grip axis is oriented transverse to the base and the shaft axis is oriented transverse to the proximal sidewall and the distal sidewall.

Implementations of the method may include one or more of the following features. The method may include thermoforming a sheet into the tray. Thermoforming the sheet into the tray may further include stretching the plurality of sidewalls to different heights to create different wall thicknesses along the length of the receptacle. The method may further include adding texture to an outer surface of the tray. Positioning the sterile surgical device in the tray in the upright orientation may include moving the sterile surgical device toward the base of the tray along the grip axis, and where the grip axis may include an upright axis.

### BRIEF DESCRIPTION OF THE DRAWINGS

The accompanying drawings, where like reference numerals may refer to functionally similar elements throughout the separate views, together with the detailed description below, are incorporated in and form part of the specification, and serve to further illustrate aspects of concepts that include the claimed disclosure and explain various principles and advantages of those aspects.

FIG. 1 is a perspective, exploded view of a packaging tray, an insert, and a cover layer, according to at least one aspect of the present disclosure.

FIG. 2 is a plan view of the packaging tray and the insert of FIG. 1, according to at least one aspect of the present disclosure.

FIG. 3 is an elevation, exploded view of the packaging tray and the insert of FIG. 1 and further depicting a surgical device positionable between the packaging tray and the insert for storage or transport, according to at least one aspect of the present disclosure.

FIG. 4 is a perspective, exploded view of a packaging tray, an insert, and a cover layer, according to at least one aspect of the present disclosure.

FIG. 5 is a perspective view of the packaging tray and the insert of FIG. 4, according to at least one aspect of the present disclosure.

FIG. 6 is a plan view of the packaging tray of FIG. 4, according to at least one aspect of the present disclosure.

FIG. 7 is an elevation view of the packaging tray of FIG. 4, according to at least one aspect of the present disclosure.

FIG. 8 is an elevation view of the packaging tray of FIG. 5 and further depicting a textured grip on the tray handle, according to various aspects of the present disclosure.

FIG. 9 is a cross-sectional elevation view of the packaging tray of FIG. 4 taken along the plane 9-9 in FIG. 7 and further depicting a surgical device positioned in the packaging tray, according to at least one aspect of the present disclosure.

FIGS. 10, 11, and 12 are cross-sectional elevation views of the packaging tray of FIG. 4 taken along the planes 10-10, 11-11, and 12-12, respectively, in FIG. 8 and depicting the cross-sectional plane through the receptacle of the tray with shading for illustrative purposes, according to at least one aspect of the present disclosure.

FIG. 13 is a perspective view of the surgical device of FIGS. 3 and 9, according to at least one aspect of the present disclosure.

FIG. 14 is a perspective view of a surgical device, according to at least one aspect of the present disclosure.

FIG. 15 is an elevation view of a pair of medical device packages within a packaging envelope, where each medical device package includes the packaging tray, the insert, and the cover layer of FIG. 1, according to at least one aspect of the present disclosure.

FIG. 16 is an elevation view of a pair of medical device packages within a packaging envelope, where each medical device package includes the packaging tray, the insert, and the cover layer of FIG. 4, according to at least one aspect of the present disclosure.

FIG. 17 is a flowchart for using a surgical device packaging tray, according to at least one aspect of the present disclosure.

The medical devices and packaging disclosed herein have been represented where appropriate by conventional symbols in the drawings, showing only those specific details that are pertinent to understanding the various aspects of the present disclosure so as not to obscure the disclosure with details that will be readily apparent to those of ordinary skill in the art having the benefit of the description herein.

### DETAILED DESCRIPTION

The present disclosure is directed to medical devices and packaging for medical devices. In the description, for purposes of explanation and not limitation, specific details are set forth, such as particular aspects, procedures, techniques, etc. to provide a thorough understanding of the present technology. However, it will be apparent to one skilled in the art that the present technology may be practiced in other aspects that depart from these specific details.

A number of factors may be considered when designing packages for medical devices. The package may be designed to hold and protect the medical device in order to avoid damage to the device while it is in storage, being transported, or during use. For example, the construction of a package for an elongated surgical device including a handle and an elongate shaft may include features to securely hold the surgical device within the package in order to prevent damage to the device itself and to the package during shipping and storage thereof.

The medical device and the packaging may also be subjected to a sterilization process (e.g. autoclaving) and, in such instances, the medical device and packaging therefor may be selected to withstand the sterilization process.

Efficient handling and opening of the packaging is desirable, as well as accessibility of the medical device within the opened package. For example, the package may be constructed to facilitate placement of the medical device within the packaging and for easy removal of the medical device from the package by a clinician without unintentional contact between the medical device and portions of the packaging (e.g. the rim of the tray). In various instances, certain contact between the medical device and portions of the packaging defeats a sterilization condition of the medical device and, thus, may necessitate a replacement device, for example.

Moreover, packages for medical devices are subject to considerations such as the amount and cost of the materials, the cost and complexity of the tooling to manufacture the packaging, ease of assembly, and ease of manufacture and transportation of the packaging and medical device therein.

Balancing the foregoing and other considerations, which are sometimes competing with each other, is difficult. For example, a reduction in packaging material (e.g. using less plastic in the packaging material) may have an effect on the durability and/or resiliency of the packaging. As another example, the addition of features to improve package handling and/or the preservation of a medical device's sterilization condition may have an effect on the manufacturing process and/or increase the weight and/or size of the packaging, which may increase manufacturing costs in certain instances.

In various aspects of the present disclosure, packaging for a surgical device includes an integral handle. A clinician may manually grasp the handle to open the package and/or to present the opened package to another clinician. For example, the clinician may firmly grasp the handle of the medical packaging to peel a cover layer from over the access opening, which may provide access to a receptacle of the medical packaging. The handle may be integrally formed with a packaging tray holding the surgical device. For example, sidewalls of the packaging tray may form the receptacle for receiving the medical device, and handle sidewalls at a deep well of the receptacle may form the handle. The handle of the packaging tray may be deflectable. For example, the handle sidewalls may be deflectable inwardly when manually grasped by a clinician. In such instances, the handle sidewalls may deflect inwardly toward opposing lateral sides of a device handle of a surgical device that is positioned in the receptacle of the tray. Under typical manual handling of the tray by assistants of average hand strength, the handle sidewalls may deflect inwardly into contact with the handle of the surgical device, such as the upright pistol grip of the handle, which may provide a secure connection between the tray and the surgical device for supporting the tray and surgical device for opening and/or presentation, for example.

As an example, a container may include a tray including a base, a rim, and sidewalls extending between the base and the rim. The base and the sidewalls may form a receptacle, and the receptacle may define a varying depth along the length thereof; the varying depth includes a peak depth intermediate a first end and a second end of the receptacle. The sidewalls may include handle sidewalls extending to the base of the tray at the peak depth of the receptacle and forming at least a portion of the tray handle. The handle sidewalls may be laterally spaced to accommodate a handle of a surgical device and sized to deflect inwardly toward opposing lateral sides of the handle of the surgical device when manually grasped by a clinician.

In various instances, a clinician may grasp the device handle using the integral handle of the tray to ground the surgical device within the tray and generate sufficient leverage to remove a cover layer from the tray. For example, the clinician may be able to grab onto the underlying surgical device handle to anchor the surgical device within the tray for opening the container and/or presenting the tray and the surgical device therein to another clinician. In such instances, the container may be easier to hold, open, and/or present to an appropriate clinician. A handle and grip integrated into the outer surface of the tray may provide a gripping surface for stabilizing the tray during aseptic transfer of the surgical device in various instances.

Additionally or alternatively, the container may ensure that the contents thereof are maintained in a sterile condition while being cost efficient and/or using less material to form the container. For example, the deep portion of the tray may correspond to the tray sidewalls that have a thinner wall thickness. Thinner sidewalls may deflect more easily than thicker sidewalls, for example, which may facilitate gripping of the device handle at the handle sidewalls of the tray to anchor the surgical device relative to the container.

Additionally or alternatively, the handle formed by the tray may be positioned at and/or near the heaviest part of the surgical device. For example, the center of mass of the surgical device may be positioned at the proximal portion of the device at or near the surgical device handle. Holding the tray and the surgical device therein at or near its center of mass provides balance and stability to the tray being held in a clinician's hand during a surgical procedure, for example.

In various instances, a surgical device may be stored or laid in a side-lying orientation, in which the surgical device lies on a lateral side thereof. More specifically, the handle of the surgical device includes a grip about which a surgeon may grip to hold the handle. The grip may be generally cylindrical and include ergonomic contours along the outer surface to comfortably accommodate a treating clinician's hand being wrapped around the grip. The handle may be designed for single-handed use. For example, a clinician may hold the handle in one hand and reach actuators (e.g. triggers) of the handle with one or more fingers of the same hand while grasping the grip and supporting the surgical device. The clinician may hold the grip with a full hand power grip, for example, which allows the clinician to grasp the outer surface of the grip against the clinician's palm and actuate an actuation trigger by pulling one or more fingers toward the clinician's palm, for example. Actuators may include an actuation trigger supported for pivotable motion relative to the grip about a pivot axis, for example. The pivot axis of such an actuation trigger may extend side-to-side or laterally across opposing lateral sides of the surgical device and the handle thereof. In the side-lying orientation of the surgical device, the pivot axis of the actuation pivot may be oriented in a vertical or upright position extending through and upward from the base of the tray.

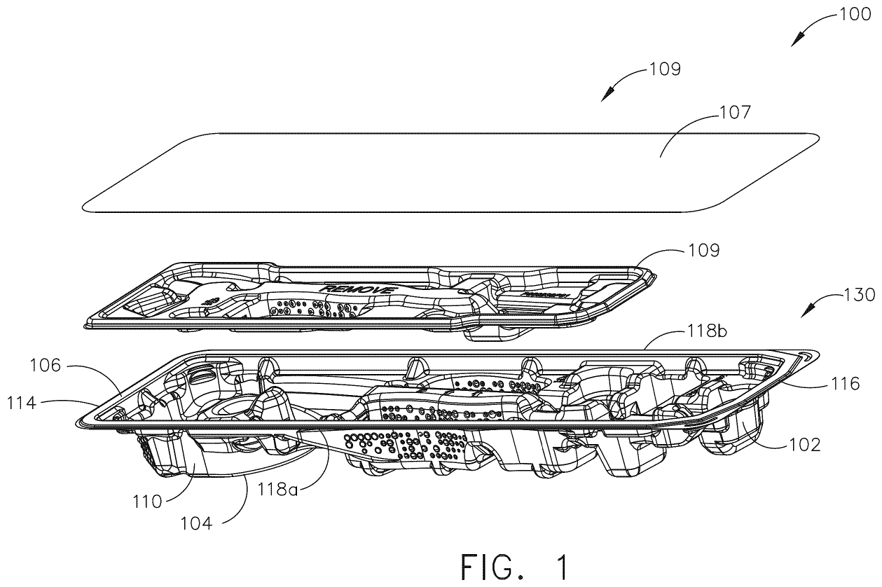

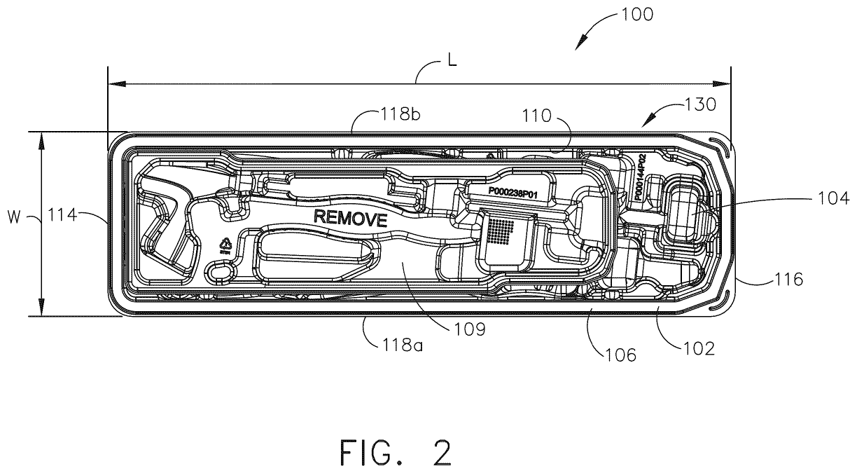

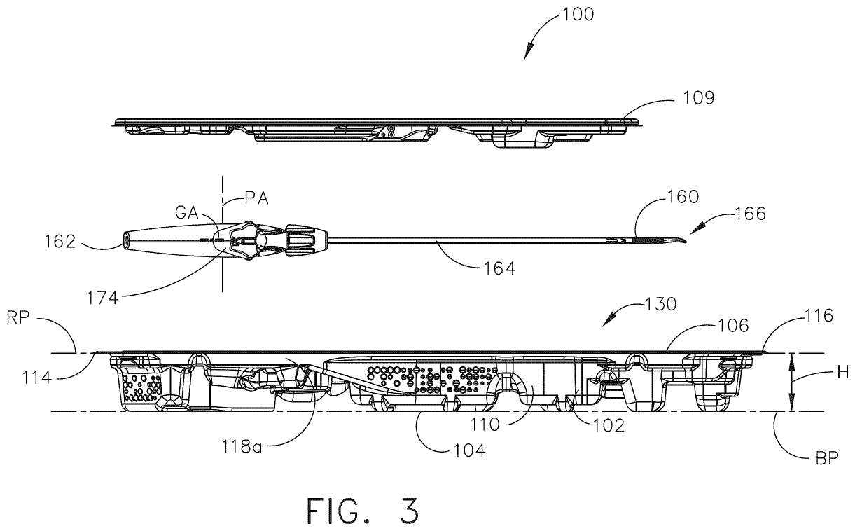

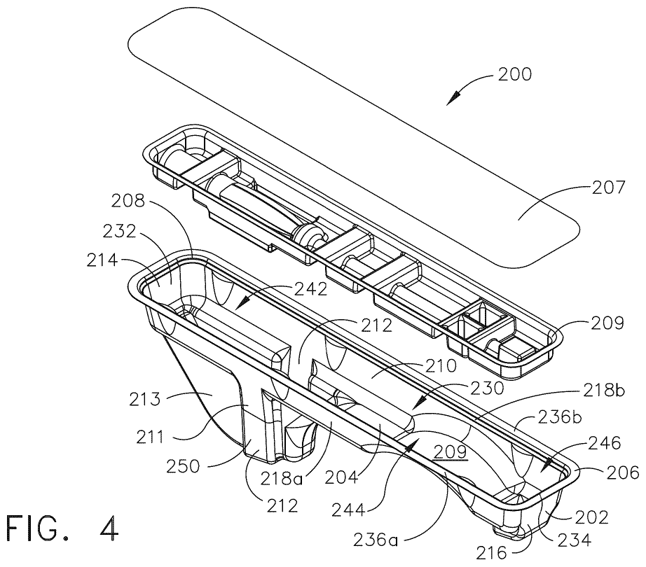

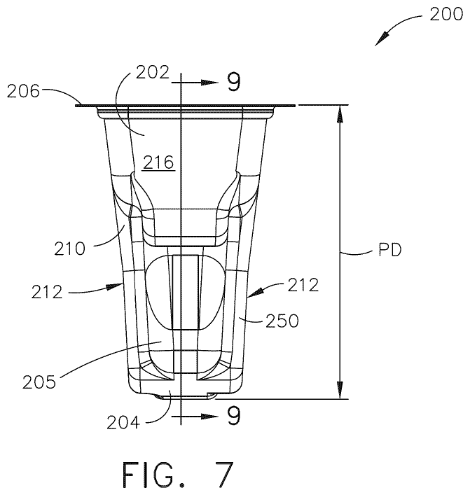

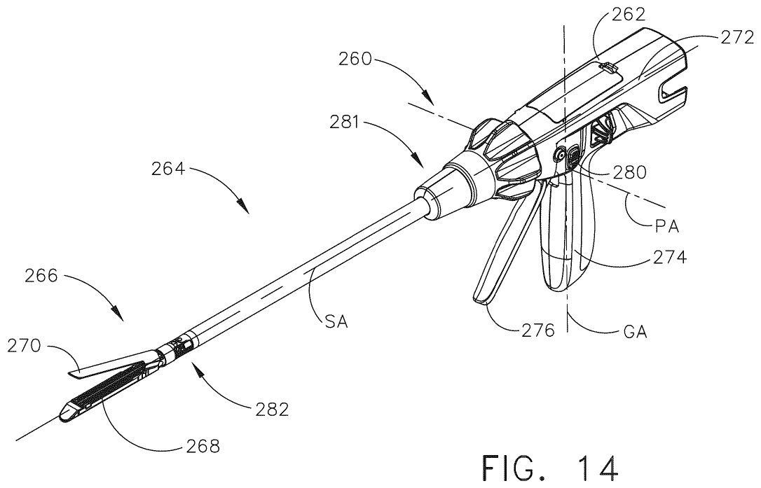

Various containers for medical devices are further disclosed herein. A container 100 for a medical device is shown in FIGS. 1-3. The container 100 is structured to receive and hold a surgical device in a side-lying orientation, for example. In contrast, a container 200 for a medical device is shown in FIGS. 4-12, and the container 200 is structured to receive a hold a surgical device in an upright orientation, for example. Exemplary surgical devices for storage within the trays 100, 200 are further disclosed herein, such as the surgical device 160 (FIGS. 3, 9, and 13) and the surgical device 260 (FIG. 14), for example.

Referring again to FIGS. 1-3, the container 100 includes a tray 102 having a base 104, a rim 106, and sidewalls 110 that extend between the base 104 and the rim 106. A cover layer 107 (FIG. 1) is removably positioned over the tray 102. The cover layer 107 may be a peelable layer releasably adhered to the rim 106 (e.g. by an adhesive and/or heat seal process). A surgical device is positionable in the tray 102 in a side-lying orientation (see, e.g. FIG. 3), and the retainer 109 is positionable over the side-lying surgical device. Thereafter, the cover layer 107 may be applied to the rim 106 to seal the surgical device and the retainer 109 within the tray 102. The sealed container 100 may provide a sterile environment for storing a sterile surgical device.

The tray 102 includes a first end 114, a second end 116, and longitudinal sides 118a, 118b extending between the first end 114 and the second end 116. The width W of the tray 102 is defined between the longitudinal sides 118a, 118b, and the length L of the tray 102 is defined between the first end 114 and the second end 116. In the depicted example, the width W is less than the length L, and the tray 102 is dimensioned to accommodate an elongate surgical device including an elongate shaft owing to the length L.

The sidewalls 110 of the container 100 form a receptacle 130 for receiving and securing components of the surgical device and, in certain instances, accessories for the surgical device. Some sidewalls 110 form an outside boundary surface of the tray 102, and other sidewalls 110 form interior dividers within the tray 102.

Referring primarily to FIG. 3, the rim 106 defines a rim plane RP, and the base 104 defines a base plane BP that is parallel to the rim plane RP. The height H of the tray 102 measured between the base plane BP and the rim plane RP is the same at numerous locations along the length of the tray 102, which provides a flat and stable surface for supporting the base 104 of the tray 102. In the depicted example, the height H is less than the width W and less than the length L, and the tray 102 is structured to accommodate an elongate surgical device (e.g. the surgical device 160) in the side-lying orientation.

As an example, the tray 102 can have a width W of about 7.4 inches, a length L of about 25.5 inches, and a height H of about 2.26 inches. In such instances, the length (I) of the inner perimeter of the rim 106 can be about 64.3 inches, and the surface area (SA) of a planar access opening into the receptacle 130 can be about 190.4 square inches. In various instances, the volume (V) of the receptacle 130 of the tray 102 can be about 186.6 cubic inches. In such instances, the ratio of the volume of the receptacle 130 to the opening surface area (V/SA) can be about 2.9 inches, for example, and the ratio of the volume of the receptacle 130 to the inside perimeter length (V/I) can be about 0.98 square inches. In various instances, the ratio of the width W to the height H can be about 3.3.

In other instances, for similar horizontal trays structured to receive a side-lying surgical device, the ratio of the volume of the receptacle 130 to the opening surface area (V/SA) can be less than 3.1 inches, less than 2.9 inches, or less than 2.5 inches, for example, and/or the ratio of the volume of the receptacle 130 to the inside perimeter length (V/I) can be less than 1.0 square inches, less than 0.9 square inches, or less than 0.8 square inches, for example. The ratio of the width W to the height H can be greater than 1, greater than 2, or greater than 3, for example. The ratios can depend on the particular tray geometry and intended surgical device for storing therein.

In various aspects, storing a surgical device in a side-lying or horizonal orientation in a tray corresponds to a larger width W, a larger inner perimeter length I, and a larger opening surface area SA than storing the same surgical device in an upright orientation, as further disclosed herein. Additionally or alternatively, storing a surgical device in a side-lying or horizonal orientation in a tray may correspond to a smaller tray height H than storing the same surgical device in an upright orientation, as further disclosed herein. Additionally or alternatively, storing a surgical device in a side-lying or horizonal orientation in a tray may correspond to a larger length L than storing the same surgical device in an upright orientation, as further disclosed herein. In other instances, upright and side-lying trays for holding the same surgical device may have about the same length L.

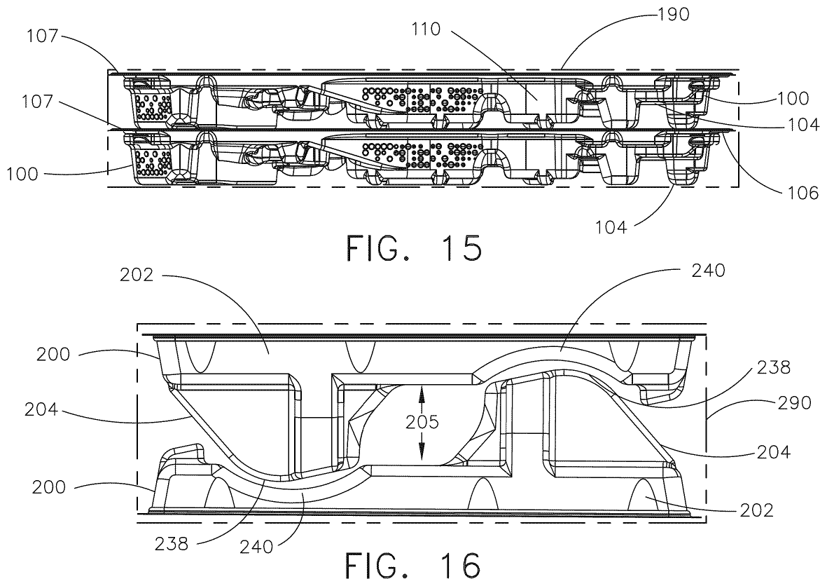

The base 104 may be positioned horizontally on a horizontal shelf or table surface, for example, and in this position the rim plane RP may extend parallel to the shelf or table surface. In one aspect, as further described with respect to FIG. 15, for example, the base 104 of one sealed container 100 may be stacked on the cover layer 107 of another sealed container 100. Owing to the rim plane RP and the base plane BP being parallel, the stacked containers 100 may form an upright column of stacked containers, in various aspects of the present disclosure.

Referring primarily to FIG. 3, when a side-lying surgical device is positioned in the container 100, such as the surgical device 160 being positioned in the side-lying orientation, for example, a lateral side of the surgical device 160 rests on an interior surface of the base 104 and the sidewalls 110 extend upwardly past the surgical device 160 to the rim 106. The lateral sides of the surgical device 160 include the lateral sides of the grip, lateral sides of the handle body, lateral sides of a shaft, and/or lateral sides of an end effector. The handle 162 of the surgical device 160 is to reside in a proximal receptacle near a first end 114 of the tray 102, and the shaft 164 of the surgical device extends distally toward a second end 116 of the tray 102.

In the side-lying orientation, the grip of the handle 162 (e.g. a generally cylindrical grip, which may be referred to as a “pistol grip” in certain instances) may extend along a grip axis GA that traverses the longitudinal sides 118a, 118b of the tray 102 and is oriented into and out of the page in the elevation view of FIG. 3. Moreover, a pivot axis PA of an actuation trigger 176 of the surgical device 160 may be oriented through the base 104 of the tray 102 and, in various instances, the pivot axis PA may be oriented in a vertical or upright position, for example. In the side-lying orientation depicted in FIG. 3, the pivot axis PA is orthogonal to the rim plane RP and the base plane BP.

The tray 102 may be formed by thermoforming, as further disclosed herein. For example, a sheet of material may be thermoformed into the tray geometry that is selected to complement an outer surface of an intended medical device and/or the accessories thereof.

To open the container 100, a clinician holds the tray 102 by an outer surface thereof and attempts to hold the tray 102 stable for removal (e.g. peeling) of the cover layer 107 from the rim 106. The outer surface of the tray 102 may not include a designated grasping region for the clinician. In certain instances, the sidewalls 110 of the tray may be difficult to grasp owing to the height H of the tray 102 and/or the spacing between the sidewalls 110. The sidewall spacing may be too wide in certain areas for the clinician to grasp in a single hand (e.g. wider than the grasping range between the thumb and fingers on one hand) and/or certain sidewalls may be inaccessible as a result of the arrangement of interior dividers formed within the receptacle 130 by the sidewalls 110. In various aspects, the space between the sidewalls 100 only accommodates a pinch grip of the sidewalls 110 between the thumb and one or more of the clinician's fingers (e.g. index and/or middle finger) rather than a full hand power grip. Without a suitable handle geometry integrated into the container 100 for grasping and supporting the tray 102, removal of the cover layer 107 and/or presentation of the surgical tool therein to a clinician may be difficult. Additional personnel and/or equipment may be utilized to support the tray 102 and open the container 100, for example.

Referring primarily to FIGS. 4-12, the container 200 is shown. The container 200 includes a tray 202 having a base 204, a rim 206, and sidewalls 210 that extend between the base 204 and the rim 206. The base 204 includes an interior surface 203 and an exterior surface 205. A cover layer 207 (FIG. 4) is removably positioned over the tray 202. For example, the cover layer 207 may be a peelable layer releasably adhered to the rim 206 (e.g. by an adhesive and/or a heat seal).

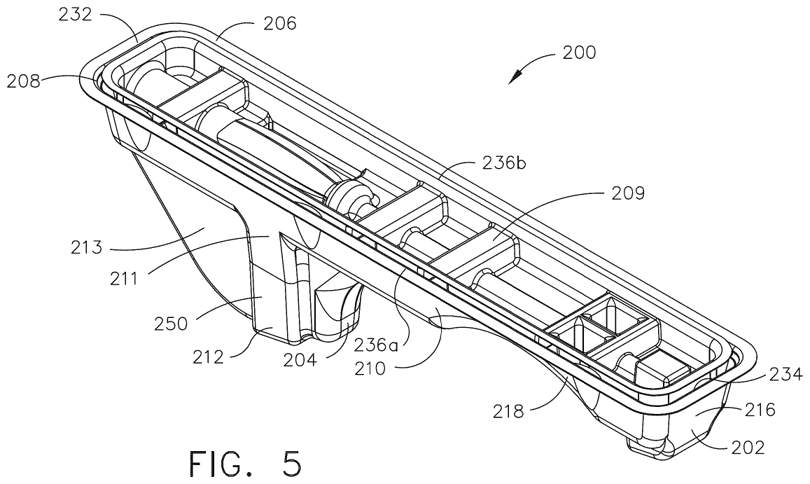

The container 200 further includes a ledge 208 that extends along an interior surface of at least a portion of the sidewalls 210 between the rim 206 and the base 204. The ledge 208 forms a shoulder for supporting an insert or retainer 209 (FIGS. 4 and 5) thereon. For example, as further disclosed herein, a medical device may be positioned in the tray 202 and the retainer 209 may be positioned over the medical device and supported along the perimeter thereof by the ledge 208. The retainer 209 may include a contoured underside structured to complement an upper surface of the medical device positioned in the upright orientation within the tray 202. Thereafter, the cover layer 207 may be applied to the rim 206 to seal the medical device within the tray 202. The sealed container 200 may provide a sterile environment for storing a sterile medical device, for example.

The sidewalls 212 include handle sidewalls 212, a proximal sidewall 214, a distal sidewall 216, and lateral sidewalls 218a, 218b positioned between the handle sidewalls 212 and the distal sidewall 216. The lateral sidewalls 218a, 218b may connect the handle sidewalls 212 to the distal sidewall 216 to form a continuous outer wall around a distal portion of the tray 202. Similarly, the handle sidewalls 212 may connect the lateral sidewalls 218a, 218b to the proximal sidewall 214 to form a continuous outer wall around a proximal portion of the tray 202.

The handle sidewalls 212 include a grip 211 and a web 213. The grip 211 is wider than the web 213 and dimensioned to receive an upright grip of a handheld surgical device therein. The walls of the grip 211 are laterally spaced apart to accommodate the upright grip of the surgical device, for example, and the walls of the web 213 may be laterally spaced to accommodate other components of the surgical device stored under the top surface of the surgical device in the receptacle, such as a coiled cable and/or device accessories, for example.

The base 204 and the sidewalls 210 form a receptacle 230, which includes a first end, or proximal end, 232 and a second end, or distal end, 234 opposite the first end 232 The receptacle 230 also includes sides 236a, 236b between the proximal end 232 and the distal end 234. The sidewalls 210 of the tray 202 form the receptacle 230 such that the first and second ends 232, 234 of the receptacle 230 correspond to the first and second ends of the tray 202, and the sides 236a, 236b of the receptacle 230 correspond to longitudinal sides of the tray 202. The inside perimeter of the rim 206 of the tray 202 defines an access opening to the receptacle 230.

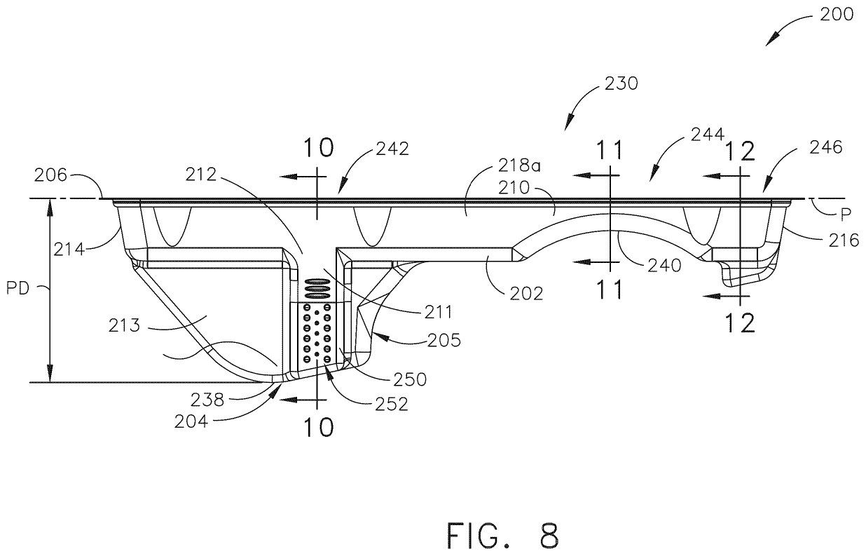

The receptacle 230 defines a varying depth between the first end 232 and the second end 234. For example, the base 204 defines an undulating surface relative to a plane defined by the rim 206 to form a plurality of wells along the length of the tray 202. Referring primarily to FIG. 8 the rim 206 defines a plane P, and the base 204 defines a contoured profile relative to the plane P. The height of the tray 202 measured from the plane P changes along the length of the tray 202. The base 204 includes a peak 238 where the tray 202 is tallest corresponding to the deepest portion of the receptacle 230. Therefore, the varying depth of the receptacle defines a peak depth PD at the peak 238, which is intermediate the first end 232 and the second end 234. The base 204 also includes a crest 240 where the tray 202 is shortest corresponding to the shallowest portion of the receptacle 230.

The crest 240 is longitudinally offset from the peak 238. For example, the crest 240 may be distal to the peak 238 such that the peak 238 receives a proximal portion of a surgical device (e.g. a handle) and the crest 240 receives a distal portion of the surgical device (e.g. an elongate shaft). In various aspects, the crest 240 forms a barrier between a handle cavity 242 and an end effector cavity 246. More specifically, the receptacle 230 includes the handle cavity 242, a shaft cavity 244, and the end effector cavity 246. The handle sidewalls 212 extend from the base 204 to the rim 206 to form a perimeter of the handle cavity 242. Moreover, the handle sidewalls 212 extend between the base 204 and the rim 206 at the peak depth PD along the varying depth of the receptacle 230. Stated differently, the handle cavity 242 is the deepest part of the receptacle 230.

The handle sidewalls 212 form a tray handle 250. A clinician may hold the tray 202 at the tray handle 250 during use. For example, the clinician may grasp the tray handle 250 when removing the cover layer 207 from the tray 202 and/or presenting the surgical device 160 positioned in the tray 202 for a surgical procedure. The tray handle 250 may be integrated with the sidewalls 210 of the tray 202. The tray handle 250 may form an elongate handle extending away from the rim 206. In various aspects, the height of the tray handle 250, e.g. the orthogonal distance between the rim 206 and the base 204, is greater than the width, e.g. the orthogonal distance between the handle sidewalls 212 and the sides 236a, 236b of the receptacle 230 at the handle cavity 242. The handle sidewalls 212 are laterally spaced to accommodate the handle of a surgical device and are sized to deflect inwardly toward opposing lateral sides of the handle of the surgical device 160 when the handle sidewalls 212 are manually grasped. Conversely, thicker sidewalls (e.g. the sidewalls 110 in FIGS. 1-3) may not deflect as easily when manually grasped and/or may not deflect inwardly to an anchor surface of the surgical device for supporting and balancing the surgical device at and/or near its center of mass, for example.

“Manually grasped” corresponds to the grasping force generated by a typical clinician grasping the tray handle 250 during a surgical procedure. For example, a clinician (e.g. a scrub nurse) manually grasping the tray handle 250 can exert a grasping force on the handle sidewalls 212 to hold and support the tray 202 and the weight of the surgical device 160 stored therein. The grasping force to support the tray 202 depends on the weight of the surgical device and the pulling force needed to remove the cover layer 209, for example.

When a clinician grasps the tray handle 250 with a full hand power grip to support the tray 202 and the surgical device 160 therein (e.g. by wrapping the clinician's palm around the tray handle 250 with the clinician's thumb on one side of the tray handle 250 and the clinician's fingers on the opposite side of the tray handle 250 to push the handle sidewalls 212 inward), the handle sidewalls 212 are deflectable inwardly toward the handle 162 of the surgical device 160. For a full hand power grip, the mean maximum grip force is about 107 pounds-force for men and about 64 pounds-force for women. Conversely, for a pinch grip (e.g. pinching the tray sidewalls 212 between the thumb and one or two fingers (index and/or middle finger), the mean maximum grip force is about 23 pounds-force for men and about 14 pounds-force for women. In such instances, manual grasping of the tray handle 250 with a full hand power grip enables the clinician to apply additional manual grip force to hold and support the tray 202 and the surgical device 160 therein.

In various instances, clinicians having below average hand strength (e.g. a maximum grip force of less than 64 pounds-force) can exert sufficient forces on the handle sidewalls 212 via a full hand power grip to deflect the sidewalls 212 inwardly toward an anchor surface of the surgical device 160.

In certain instances, the handle sidewalls 212 may be deflectable inwardly under a clinician's full hand power grip force but not by the same clinician's maximum pinch grip force owing to the difference between maximum full hand power grip force and maximum pinch grip force for an individual.

In various instances, a clinician may initially grasp the handle sidewalls 212 without deflecting the handle sidewalls 212 into engagement with an anchor surface of the surgical device 160. In such instances, additional manual grasping forces on the tray handle 250 by the clinician, such as to facilitate removal of the cover layer 209 away from the rim 206, can deflect the handle sidewalls 212 inwardly to the anchor surface of the surgical device 160.

In various instances, the thickness of the sidewalls 210 varies around the perimeter of the receptacle 230 and certain sidewalls (e.g. the handle sidewalls 212) are deflectable inward to an anchor surface of the surgical device 160 when manually grasped by a clinician and other sidewalls (e.g. the lateral sidewalls 218a, 218b at the shaft cavity 244) are not deflectable inwardly to an anchor surface of the surgical device 160 when manually grasped by the clinician.

The flexibility of the sidewalls 210 may be a function of the thickness of the sidewalls. For example, the handle sidewalls 212 may be more flexible than the proximal sidewall 214, the distal sidewall 216, and/or the lateral sidewalls 218a, 218b owing to the comparatively thinner wall thickness of the handle sidewalls 212.

In various aspects, a manual grasping force by a clinician holding and opening the tray 202 can be sufficient to deflect the handle sidewalls 212 inwardly and the same force may be insufficient to deflect the lateral sidewalls 218a, 218b inwardly at the cross-sectional planes 11-11 or 12-12 in FIG. 8, for example.

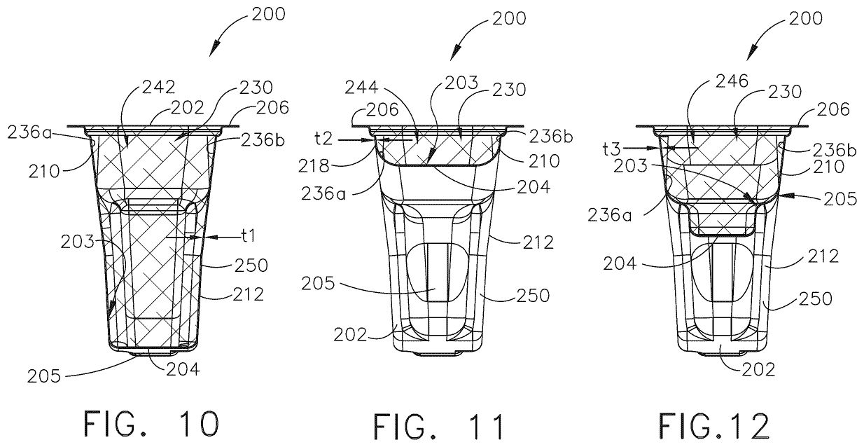

Referring primarily to FIGS. 10-12, the sidewalls 210 define different thicknesses around the perimeter of the receptacle 230. For example, the thickness of the sidewalls 210 may change along the length L of the receptacle 230. In various aspects, the thickness of the sidewall 210 may depend on the depth along that portion of the receptacle 230. For example, the sidewalls 210 may be thinnest at the peak depth PD of the receptacle 230. In such instances, the handle sidewalls 212 may be thinner than the proximal sidewall 214, the distal sidewall 216, and the lateral sidewalls 218a, 218. The handle sidewalls 212 define a thickness t1 (FIG. 10) and the lateral sidewalls 218a, 218b define a thickness of t2 (FIG. 11) at the shaft cavity 244 and a thickness of t3 (FIG. 12) at the end effector cavity 246. The thickness t1 may be less than the thickness t2 and t3. Moreover, owing the geometry of the receptacle 230, the thickness t3 may be less than the thickness t2 because the end effector cavity 246 is deeper than the shaft cavity 244 at the crest 240. For example, the receptacle 230 is shallowest at the crest 240 and, thus, the lateral sidewalls 218a, 218b bordering the shaft cavity 244 at the crest 240 may define the thickest walls.

The cross-sectional area of the cavity 230 in the plane 10-10 in FIG. 10 is greater than the cross-sectional area of the cavity 230 in the plane 11-11 in FIG. 11 and in the plane 12-12 in FIG. 12. Moreover, the cross-sectional area of the cavity 230 in the plane 11-11 in FIG. 11 is less than the cross-sectional area in the plane 10-10 and in the plane 12-12. In various aspects, the cross-sectional area through the cavity 230 along an upright plane perpendicular to the plane P (FIG. 8) at a location along the length L of the tray 202 is greatest in the handle cavity 242, for example.

In FIGS. 10-12, the tray 102 and the receptacle 130 are symmetrical about a central axis such that the sidewalls 210 have the same geometry and thickness on opposite sides of the axis of symmetry, for example. In other aspects of the present disclosure, the tray and/or receptacle may be non-symmetrical about the central axis at one or more locations along the length thereof to accommodate an asymmetry and/or additional features and/or accessory for the surgical device stored therein.

In various aspects, the wall thickness of the sidewalls 210 may vary along the height thereof, however, the minimum wall thickness and/or the average wall thickness may be minimized where the depth of the receptacle is maximized.

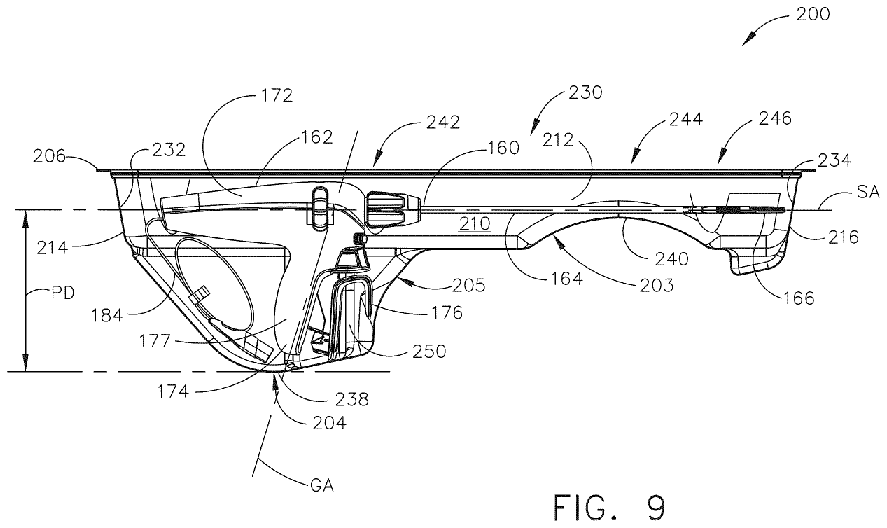

In certain instances, referring primarily to FIG. 9, the tray handle 250 may include a textured grip 252. For example, the handle sidewalls 212 forming the tray handle 250 include an outer surface, which is grasped by the clinician. The outer surface may include a texture, such as ridges, bumps, and/or dimples, for example, to increase the frictional coefficient of the outer surface and improve the clinician's ability to grasp the tray handle 250.

The tray 202 and the tray handle 250 thereof may accommodate right-handed and left-handed clinicians when opening and dispensing the surgical device in the sterile field. For example, the tray handle 250 being positioned on one side of the tray 202 enables the tray handle 250 to either be supported in the right hand or the left hand of the clinician while the other hand can peel the cover layer 207 off of the rim with the other hand.

In various aspects of the present disclosure, a surgical device may be oriented in an upright orientation in a packaging tray. In the upright orientation, the grip of the surgical device may extend along an upright axis, which may extend through the base of the packaging tray. Moreover, the shaft of the surgical device may extend along a longitudinal axis, which may extend through proximal and distal sidewalls of the packaging tray.

In one example, referring primarily to FIG. 9, the container 200 may include a surgical device, such as the surgical device 160, for example, positioned in the receptacle of the tray. The surgical device 160 includes the handle 162 and the shaft 164 extending from the handle 162 along the shaft axis SA. The handle 162 may include a grip 174 extending along a grip axis GA oriented transverse to the shaft axis SA, and the grip axis GA may extend through the base 204 of the tray 202 to orient the handle 162 in the upright orientation in the receptacle 230. The surgical device 160 is exemplary and alternative surgical devices are contemplated such as a stapling instrument 260 (FIG. 14) among others, as further disclosed herein.

In various instances, the upright orientation of the surgical device 160 positioned in the receptacle 230 may reduce the perimeter length defined around the access opening to the receptacle 230 relative to the volume of the tray 202. Stated differently, for a side-lying receptacle, such as the receptacle 130 (FIGS. 1-3), for example, dimensioned to receive the surgical device 160 in the side-lying orientation, the perimeter length of the receptacle 130 may be larger than an upright receptacle, such as the receptacle 230 (FIGS. 4-12), for example, dimensioned to receive the same surgical device 160 in an upright orientation.

Additionally or alternatively, the upright orientation of the surgical device 160 positioned in the receptacle 230 may reduce the surface area of the access opening relative to the volume of the tray 202. Stated differently, for a side-lying receptacle, such as the receptacle 130 (FIGS. 1-3), for example, dimensioned to receive the surgical device 160 in a side-lying orientation, the surface area of the access opening may be larger than an upright receptacle, such as the receptacle 230 (FIGS. 4-12), for example, dimensioned to receive the same surgical device 160 in an upright orientation.

A larger perimeter length and/or larger surface area of the access opening may correspond to additional surface area for the seal (e.g. a heat seal or adhesive) between the cover layer and the tray, which may increase the force and/or range of motion needed to remove the cover layer and expose or present the surgical device therein.

Additionally or alternatively, the upright orientation of a surgical device in the receptacle 230 may provide a deeper receptacle, which may be further structured to receive additional components of the surgical device. For example, a cord of the surgical device may be coiled and stored in an upright position adjacent to the device handle. In such instances, the coiled cord may be managed and contained within the tray even when the cover layer has been removed.

Additionally or alternatively, the upright orientation of the surgical device in the receptacle 230 can allow a clinician to visually see and identify the product from both lateral sides of the packaged container 200. For example, the sidewalls 210 of the tray 202 can be transparent or semi-transparent such that the clinician can visualize the surgical device held within the tray 202 from both lateral sides. In particular, the clinician may be able to see both sides of the handle of the surgical device.

Referring primarily to FIG. 9, the tray 202 defines the peak depth PD. As further disclosed herein, the tray 202 is adapted to receive a surgical device, such as the surgical device 160, for example, in an upright orientation with the grip axis GA of the grip 174 extending toward the base 204 of the receptacle 230. The grip axis GA extends toward the peak 238 of the receptacle 230. For example, the grip axis GA may be generally aligned with the peak 238 such that the tallest portion of the handle 162 overlies the peak 238.

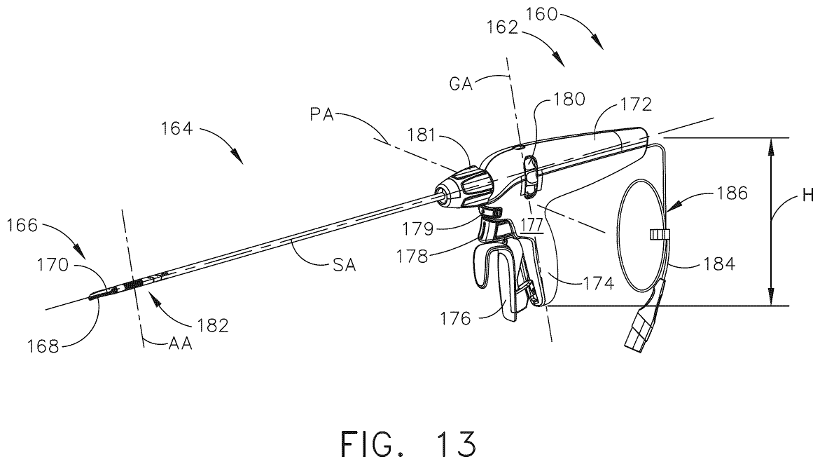

The exemplary surgical device 160 is further shown in FIG. 13. The surgical device 160 can be stored in one of the containers 100, 200. For example, the surgical device 160 can be stored in a side-lying orientation in the container 100, and in an upright orientation in the container 200. The surgical device 160 is a handheld electrosurgical instrument operable to grasp, cut, seal, and/or weld tissue, such as a blood vessel, for example. The surgical device 160 includes the handle 162, the elongate shaft 164 extending from the handle 162, and the end effector 166. The end effector 166 includes a first jaw 168 and a second jaw 170. One or both of the jaws 168, 170 can be movable to grasp, clamp, and/or energize the patient's tissue.

The handle 162 includes a body 172 and the grip 174, which is sized and structured to be held by a clinician. For example, the grip 174 may be a generally cylindrical handle, which a clinician may grasp in a single hand, as further disclosed herein. While the term “cylindrical” is used, the grip 174 need not resemble a perfect cylinder. In certain instances, the grip 174 may be referred to as a pistol grip. The grip 174 extends away from the body 172 along the grip axis GA, which is oriented transverse to the shaft axis SA defined by the elongate shaft 164. The grip 174 includes an outer surface defining a perimeter of the grip 174. The perimeter includes a pair of opposed lateral sides 177.

The handle 162 further includes at least one actuator that is actuatable by the clinician to operate the surgical device 160. The actuators encompass a jaw closure trigger 176, a knife trigger 178, an energy activation button 179, an articulation control 180, and/or a shaft rotation knob 181. Alternative actuators are also contemplated. The actuators are movable relative to the grip 174, for example, to actuate a surgical function of the surgical device 160.

The elongate shaft 164 extends distally from the handle 162 along the shaft axis SA and is rotatable relative to handle 162 about the shaft axis SA. The grip axis GA may be orthogonal and/or angularly-oriented relative to the shaft axis SA. When the elongate shaft 164 is rotated relative to the handle 162, the end effector 166 rotates with the elongate shaft 164. The handle 162 comprises the rotation actuator, e.g. shaft rotation knob 181, which is mounted to the elongate shaft 164 and is rotatable relative to the body 172 of the handle 162 by the clinician to rotate the shaft 164 about the shaft axis SA.

In various embodiments, the surgical device 160 includes a motor-driven system that is operable to rotate the elongate shaft 164 The motor-driven system may include a trigger, such as a switch, for example, accessible by the clinician operating the surgical device 160. In at least one such embodiment, the trigger is positioned on the handle 162.

The end effector 166 may be rotated relative to the shaft 164 about an articulation joint 182. The articulation joint 182 defines an articulation axis AA about which the end effector 166 may be articulated relative to the shaft 164. The articulation joint 182 also defines a plane within which the end effector 166 is articulated relative to the shaft 164. In various aspects, the shaft axis SA corresponds to the axis of a proximal segment of the shaft 164 that is proximal to the articulation joint 182 and/or to the axis of the shaft 164 in a non-articulated position. In other words, in an articulated position, a distal segment of the shaft 164 may deviate from the shaft axis SA. The surgical device 160 may further include an articulation drive system coupled to the articulation control. The articulation control 180 is positioned on the handle 162, but could be positioned in any suitable location on the surgical device 160.

In various embodiments, the surgical device 160 includes a motor-driven system that is operable to articulate the end effector 166 relative to the shaft 164. The motor-driven system may include an electric motor operable to drive the articulation driver longitudinally and a motor control circuit.

In various other embodiments, the articulation joint 182 defines more than one articulation axis, such as two articulation axes, which may be orthogonal to each other, for example, about which the end effector 166 may be rotatable relative to the shaft 164 In at least one embodiment, the articulation joint 182 includes a flexible articulation region. In still other instances, the surgical device 160 may not include an articulation joint.

In various instances, the jaws 168, 170 are movable to grasp and clamp tissue in the end effector upon actuation of the jaw closure trigger 176, which is rotatably coupled to the grip 174 of the handle 162. For example, the jaw closure trigger 176 includes a lever, which may be pulled or rotated to move the lever from an unactuated position to an actuated position and to transmit a force applied by a clinician to a closure drive. When the jaw closure trigger 176 is pulled toward the grip 174 by the clinician, the jaw closure trigger 176 may rotate about a pivot axis PA to effect closure of the jaws 168, 170.

In this example, the end effector 166 is configured to seal or weld tissue by applying bipolar radio frequency (RF) energy to tissue. However, the surgical device 160 may be configured to seal or weld tissue through any other suitable means. For example, the surgical device 160 may be configured to seal or weld tissue via an ultrasonic blade, staples, etc.

The surgical device 160 is electrically coupled to a power source via a cord or power cable 184. The power source may be configured to provide all or some of the electrical power requirements for the surgical device 160. The power cable 184 may be wound in a coil 186 for storage and shipping. In other instances, the surgical device 160 may contain an internal power source or plurality of power sources, such as a battery and/or supercapacitors.

A surgical device like the surgical device 160, for example, is further disclosed in, for example, U.S. Patent Application Publication No. 2025/0040982 to Otrembiak et al., titled ARTICULATION SECTION FOR SHAFT ASSEMBLY OF SURGICAL INSTRUMENT, which published Feb. 6, 2025 and is incorporated by reference herein in its entirety.

Referring again to FIG. 9, the surgical device 160 may be positioned in the receptacle 230. The surgical device 160 may be sterilized and the container 200 may maintain the sterile condition of the surgical device 160. For example, the surgical device 160 may be enclosed or encased in the container 200 between the tray 202 and the cover layer 207 (FIG. 4).

The shaft axis SA of the surgical device 160 is oriented along the length L of the tray 202 and the lateral width of the surgical device 160 is positioned between the lateral sidewalls 218a, 218b of the receptacle 230. In the upright orientation, the grip axis GA extends through the base 204 of the tray 202. The peak depth PD may be greater than the width W and less than the length L of the tray. The increased depth of the receptacle 230 is dimensioned to receive the handle 162 including the grip 174 thereof in an upright orientation. The handle 162 may further include a height H, and the peak depth PD may be greater than the height H. In such instances, an upper edge of the handle 162 is positioned below the rim 206 of the tray 202 when the surgical device 160 is positioned in the receptacle 230. The perimeter of the grip 174 is surrounded by a subset of the sidewalls 210. For example, the handle sidewalls 212 are adjacent to the pair of opposing lateral sides 177 of the grip 174.

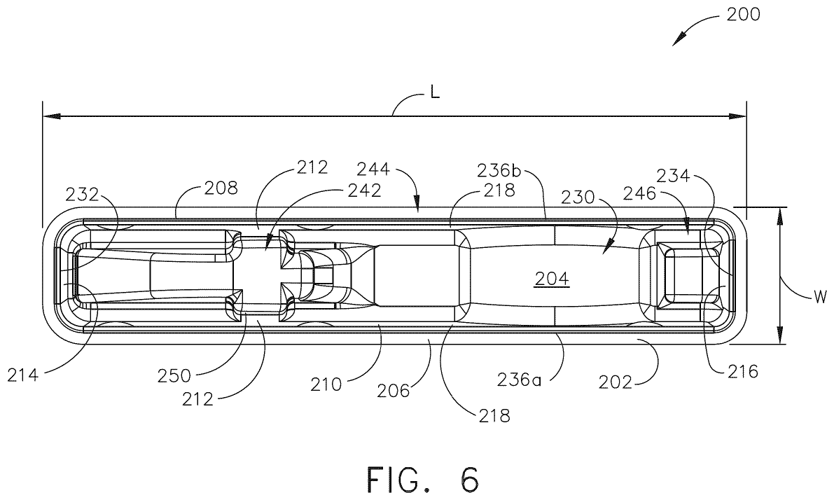

Referring primarily to FIG. 6, for a surgical device in the upright orientation, the width W of the tray 202 may provide sufficient lateral clearance around the elongate shaft 164 of the surgical device 160. For example, the width W of the tray 202 is sufficiently wide to accommodate the opposing lateral sides 177 of the grip 174, which is wider than the elongate shaft 164. In various instances, the increased clearance around the elongate shaft 164 within the shaft cavity 244 of the tray 202 may more comfortably accommodate the digits (e.g. fingers) of the clinician reaching into the shaft cavity 244 to withdraw the surgical device 160 from the tray while avoiding contact with the rim 206 of the tray 202.

In various aspects, the crest 240 of the base 204 is positioned to support the shaft 164 of the surgical device 160 at a location intermediate the handle 162 and the end effector 166. The distal cavity 246 of the receptacle 230 is dimensioned to receive the end effector 166. The end effector 166 may be suspended or able to float in the distal cavity 246. For example, the handle 162 and the shaft 164 may be sandwiched and supported between the base 204 and the retainer 209 and/or the cover layer 207 to hold the handle 162 and the shaft 164 in position relative to the receptacle 230.

The contours of the tray 202 and the retainer 209 are structured to securely hold portions of the surgical device 160 therebetween. The surgical device may include datum surfaces or contours, such as portions of an upper surface, rear surface, bottom surface, and/or lateral side surface of a handle casement, for example, that are received and retained by corresponding datums of the tray 202 and/or the retainer 209. Certain datum surfaces may be the same across different surgical devices. In such instances, different types of surgical devices can be used with the same container. Even surgical devices for entirely different functions may have a similar handle profile and/or shaft length, for example, and may be stored and transported in the same container.

Accessories for the surgical device 160 may be positioned in the receptacle 230, which securely holds the accessories even when the retainer 209 is removed. For example, the coil 186 of power cable 184 may be stored in the receptacle 230 along with the surgical device 160. The power cable 184 may be oriented in an upright orientation generally parallel to the grip axis GA. In various aspects, the web 213 of the handle sidewalls 212 may be positioned securely around the coil 186 to hold the power cable 184 in the coiled orientation even when the cover layer 207 and/or the retainer 209 have been removed from the tray 202. An upright orientation of the coil 186 below the rim 206 may further help to prevent inadvertent contact between the coil 186 and the rim 206, for example.

The surgical device 160 is exemplary of surgical devices that can be stored and transported in the containers 100, 200. A further example is the stapling instrument 260 depicted in FIG. 14, for example. The stapling instrument 260 includes a handle 262, an elongate shaft 264 extending distally from the handle 262, and an end effector 266 extending distally from the elongate shaft 264. Similar to the handle 162, the handle 262 includes a body 272, a grip (e.g. a pistol grip) 274, and at least one actuator. For the stapling instrument 260, the actuators encompass a jaw closure trigger 276, an articulation switch 280, and/or a shaft rotation knob 281, for example. One or more additional actuators are contemplated, such as a separate actuator for clamping and/or firing staples, for example. An internal power source (e.g. battery pack) can be releasably attached to the handle 262, such as to the body 272, for example. The end effector 266 includes a staple cartridge jaw 268 and an anvil jaw 270 rotatable relative to the staple cartridge jaw 270 to clamp tissue therebetween. In other aspects of the present disclosure, both jaws 268, 270 and/or just the staple cartridge jaw 268 may be rotatable to open and close the end effector 266. Stapling instruments like the stapling instrument 260, for example, are further disclosed in, for example, U.S. patent application Ser. No. 18/379,790 to Fiebig et al., titled METHOD OF ASSEMBLING A STAPLE CARTRIDGE, filed Oct. 13, 2023, which is incorporated by reference herein in its entirety.

Referring again to FIGS. 4-12, the tray 202 can have a width W of about 4.46 inches, a length L of about 22.9 inches, and a height H of about 6.07 inches. In such instances, the inner perimeter length (I) of the rim 206 in the rim plane P (FIG. 8) can be about 52.7 inches, and the surface area (SA) of a planar access opening into the receptacle 230 can be about 100.9 square inches. In various instances, the volume (V) of the receptacle 230 of the tray 202 can be about 164.8 cubic inches. In the foregoing example, the ratio of the volume of the receptacle 230 to the opening surface area (V/SA) is about 1.6 inches, for example, and the ratio of the volume of the receptacle 230 to the insider perimeter length (V/I) is about 3.1 inches. In various instances, the ratio of the width W to the height H can be about 0.7

In other instances, for similar upright trays structured to receive an upright surgical device, the ratio of the volume V of the receptacle 230 to the opening surface area (V/SA) can be greater than 2.9 inches, greater than 3.1 inches, and/or greater than 3.5 inches, for example, and/or the ratio of the volume of the receptacle 230 to the inside perimeter length (V/I) can be greater than 1.0 square inches, greater than 1.5 square inches, or greater than 2 square inches, for example. The ratio of the width W to the height H can be less than 0.7, less than 1, or less than 2, for example. For example, the tray 202 is taller than its width, whereas the tray 102 is wider than its height. The ratios can depend on the particular tray geometry.

In various aspects, storing a surgical device in an upright orientation in a packaging tray can correspond to a narrower width W, a smaller inner perimeter length I, and a smaller opening surface area SA than storing the same surgical device in a side-lying orientation. For example, the width of a tray can be reduced by about 50%. In other aspects, the width of a tray can be reduced by more than 50%, such as by more than 60% or more than 70%, for example, and, in other instances, by less than 50%, such as by less than 25% or less than 40%, for example. Moreover, the inner perimeter length of the access opening can be reduced by about 20%. In other aspects, the width of a tray can be reduced by more than 20%, such as by more than 25%, more than 35%, or more than 50%, for example, and, in other instances, by less than 20%, such as by less than 10% or less than 15%, for example. Moreover, the opening surface area can be reduced by about 60%. In other aspects, the opening surface area of a tray can be reduced by more than 60%, such as by more than 70% or more than 80%, for example, and, in other instances, by less than 60%, such as by less than 25%, less than 40%, or less than 50%, for example.

Additionally or alternatively, storing a surgical device in an upright orientation in a packaging tray can correspond to a larger height H than storing the same surgical device in a side-lying orientation. For example, the height of the tray can be increased by about 90%. In other aspects, the height of the tray can be increased by less than 90%, such as by less than 50% or less than 75% and, in other instances, by more than 90%, such as by more than 100% or more than 120%, which is more than twice as tall, for example.

Additionally or alternatively, storing a surgical device in an upright orientation in a packaging tray can correspond to a shorter length than storing the same surgical device in a side-lying orientation. For example, the length of the tray can be reduced by about 10%. In other aspects, the length of the tray can be reduced by more than 10%, such as by 15% or 20%, for example, and, in other instances, by less than 10%, such as by 2% or 5%, for example. In other instances, upright and side-lying trays for holding the same surgical device can have about the same length L or the upright tray may be longer than a side-lying tray for the same surgical device.

For the same surgical device, e.g. the surgical device 160 (FIG. 13) or the stapling instrument 260 (FIG. 14), a tray structured to hold the surgical device in an upright orientation may reduce the ratio of the volume V of the receptacle to the opening surface area (V/SA) by more than 50%, and may reduce the ratio of the volume V of the receptacle to the inside perimeter length of the rim by more than 5%, for example.

In certain aspects, less force may be required to remove the cover layer 207 (e.g. a flexible film) from the rim 206 than to remove the cover layer 107 from the rim 106 owing to the reduced perimeter length and/or reduced surface area of the access opening. Additionally or alternatively, a smaller range of motion may be needed to remove the cover layer 207 from the rim 206 than to remove the cover layer 107 from the rim 106.

In various aspects of the present disclosure, a set or pair of packaging containers for surgical devices may be nested base-to-base for storage and/or transportation. For example, the base of a packaging tray may define a varying depth where the peak depth along the length of the packaging tray is structured and sized to receive the tallest portion of the surgical device, e.g. the handle of the surgical device oriented in an upright position. The orientation of one of the packaging trays may be flipped and rotated relative to the other packaging tray in the pair or set such that the outward-facing surfaces of the tray bases nest together base-to-base for storage and/or transport. The cover layer for each container is positioned and structured to secure the surgical device and, in various instances, accessories therefor, within the receptacle regardless of the orientation of the package. For example, with the surgical device retained therein, the tray may be positioned on the base, the cover layer (which may be supported by the rim or the tray), or a lateral side of the tray.

In one example, a packaging set includes two surgical device packaging trays and each tray includes a base and sidewalls extending from the base to form a surgical device receptacle. Each base may include a first outward-facing surface including a first protrusion and a first indentation. The two surgical device packaging trays may be oriented such that the outward-facing surfaces face each other and the protrusion of each outward-facing surface nests in the indentation of the other outward-facing surface.

In certain instances, such a packaging set may reduce the size of the packaging envelope and/or the unoccupied space within the packaging envelope. For example, the packaging set may be stored and transported in a close-fitting rectangular carton defining the packaging envelope. Close-fitting cartons may slidably receive the packaging set and hold the containers of the packaging set relative to each other to prevent movement of the containers relative to the carton and each other during shipping. The containers may be removable from the close-fitting cartons by gravity upon tipping of an open carton to release the containers therefrom.

A packaging set having nested base-to-base containers may assume a smaller volume carton than a packaging set comprised of stacked surgical device trays (e.g. where the base of one surgical device tray is stacked on the cover of the other surgical device tray) for the same surgical device. Additionally or alternatively, the packaging set may have less unoccupied space within the carton than a packaging set comprised of stacked surgical device trays for the same surgical device. Smaller packaging envelopes and/or less unoccupied space may correspond to more efficient shipping and storage of the surgical devices in certain instances. For example, containers storing a surgical device in an upright orientation may occupy less shelf space and/or reduce the sterilization volume, which may be limited in certain sterilization chambers, such as ethylene oxide and/or gamma radiation sterilization chambers having limited capacities.

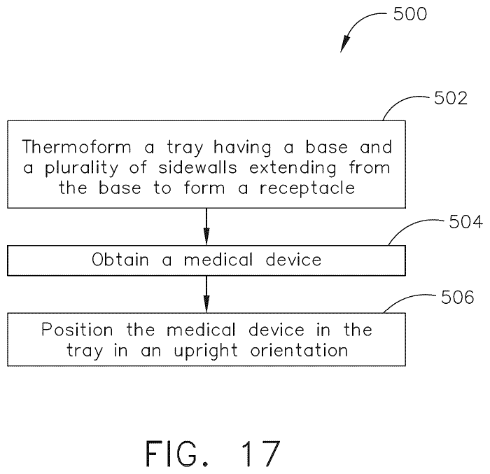

Referring primarily to FIG. 16, an exemplary packaging envelope 290 for a set of containers 200 is shown. The set of containers includes paired containers (e.g. one or more sets of two containers 200). The outward-facing or exterior surface 205 of each base 204 extends along the length of the tray 202. The base 204 includes the peak 238, which forms a protrusion along the exterior surface 205, and further includes a crest 240, which forms an indentation along the exterior surface 205. The exterior surfaces 205 of the containers 200 are configured to face each other when surgical devices are stored in the respective containers 200. For example, the protrusion of one exterior surface 205 is structured and sized to nest in the indentation of the other exterior surface 205 and vice versa.

The set of containers 200 (FIGS. 4-12) may further include a carton, which may be a rectangular box that is dimensioned to securely receive the nested base-to-base containers 200. For example, the set of containers 200 may be closely received in the carton to minimize movement of the containers within the sealed carton. The carton may correspond to the packaging envelope 290 for the pair of containers 200. In an example, the packaging envelope 290 for the carton may define a volume of about 775 cubic inches. In various aspects, less than 50% of the carton volume may be unoccupied by the set of containers 200, which may be loaded with surgical devices for storage and transport, or example. For example, about 42% of the carton volume may be unoccupied by the set of containers 200 nested base-to-base in the carton and loaded with surgical devices. In other instances less than 42% of the carton volume may be unoccupied by the set of nested containers 200, such as less than 40% or less than 30% and, in other instances, more than 42% of the carton volume may be unoccupied by the set of nested containers 200, such as more than 45%, more than 50% or more than 60%, for example.

Conversely, referring primarily to FIG. 15, the containers 100 (FIGS. 1-3) are stored in a carton in a stacked orientation in which the base 104 of one tray 102 is stacked on the cover layer 107 of another container 100. In such instances, the pair of stacked containers 100 may define a packaging envelope 190 for a carton having a volume of 869.6 cubic inches. In various aspects, less than 50% of the carton volume may be unoccupied by the set of containers 100, which may be loaded with surgical devices for storage and transport, or example. For example, about 42% of the carton volume may be unoccupied by the set of containers 100 stacked base-to-rim in the carton and loaded with surgical devices. In other instances less than 42% of the carton volume may be unoccupied by the set of stacked containers 100, such as less than 40% or less than 30% and, in other instances, more than 42% of the carton volume may be unoccupied by the set of stacked containers 100, such as more than 45%, more than 50% or more than 60%, for example.

In various aspects, packing a pair of upright surgical device containers base-to-base in a shipping carton, such as the containers 200 in FIG. 16, for example, takes up less volume than packing a pair of stacked surgical device containers, such as the containers 100 in FIG. 15, for example, for the same surgical device. For example, the volume of the packaging envelope can be reduced by about 12% (e.g. from about 869.6 cubic inches for two stacked containers 100 to about 775 cubic inches for two nested containers 200). In other aspects, the volume of the packaging envelope can be reduced by more than 12%, such as by more than 15%, more than 20%, or more than 25%, for example, and, in other instances, by less than 12%, such as by less than 5% or less than 10%, for example.

In various aspects, the containers disclosed herein (e.g. containers 100, 200) are manufactured by a thermoforming technique. For example, the manufacturing method may utilize thermoforming of the receptacle in the tray for the surgical device where the deepest portion of the receptacle is structured to receive an upright handle of the surgical device. The method may further include positioning a sterile surgical device in the receptacle in an upright orientation. The thermoformed receptacle may have a varying depth and varying sidewall thicknesses, as further disclosed herein.

In one example, a method may include positioning a sterile surgical device in a tray in an upright orientation, wherein, in the upright orientation, a grip axis of the handle of the sterile surgical device is oriented transverse to a base of the tray and the shaft axis of the elongate shaft of the sterile surgical device is oriented transverse to proximal and distal sidewalls of the tray. The method may further include thermoforming the tray from a sheet.

The thermoformed sheet for the tray is comprised of a plastic, such as polyethylene terephthalate (PETG), for example. Alternative plastics for the tray are contemplated including polyesters, amorphous polyesters, copolyesters, polystyrenes, PTEG foams, polypropylenes, polycarbonates, and/or high-density polyethylenes, for example. The material of the tray is sufficiently rigid to support the cantilevered load of the tray and the surgical device positioned therein when held at and/or near an end of the tray and/or at the integrated tray handle, for example.

In various instances, such packaging for a surgical device oriented in an upright orientation may use a smaller sheet of raw material than packaging for the same surgical device oriented in a side-lying orientation. Additionally or alternatively, the packaging may reduce the amount of packaging material (e.g. use less plastic to hold the same surgical device). For example, the packaging tray 202 may use up to 50% less material (e.g. less plastic) than the packaging tray 102. In other aspects, the tray 202 may reduce the amount of material by more than 20%, more than 40% or more than 50% compared to the tray 102, for example. The amount of material depends on the surface area and the thickness of the sheet of material.

For example, the packaging tray 102 may be thermoformed from a sheet of material having a surface area of about 190 square inches, and the packaging tray 202 may be thermoformed from a sheet of material having a surface area of about 100 square inches. The foregoing sheets of material correspond to a 44% reduction in surface area to form the packaging tray 202 in contrast to the packaging tray 102, and a 44% reduction in material in instances in which the sheets have the same thickness. In other instances, a thicker sheet may be used to form the packaging tray 202. For example, a thicker sheet may be used to form the deeper receptacle, for example. In such instances, a reduction in material may still be achieved based on the smaller surface area of the sheet. For example, a 30% reduction in material correspond to a thicker sheet of material being used to form the packaging tray 202 in contrast to a thinner sheet of material being used to form the packaging tray 102. In certain instances, a reduction in packaging material may lead to less medical waste being generated during surgical procedures.

In various aspects, the packaging may have a greater depth and thinner sidewalls at the portion of the receptacle that is structured and dimensioned to receive the upright grip portion of a surgical device handle. The thinner sidewalls may be more flexible and deflectable inwards toward the upright grip portion positioned therein to facilitate grasping of the upright grip portion through the thinner sidewalls of the tray, for example.

Referring primarily to FIG. 17, a method 500 is shown. The method 500 may be used to form the container 200, for example. The method 500 includes, at step 502, thermoforming a sheet into the tray, where the tray includes a base and sidewalls extending from the base to form the receptacle. The sidewalls include a proximal sidewall and a distal sidewall and a longitudinal axis of the tray extends between the proximal sidewall and the distal sidewall. In other instances, the method 500 includes obtaining a thermoformed tray. The method 500 further includes, at step 504, obtaining a medical device, such as a surgical device (e.g. surgical device 160 in FIG. 13 or the stapling instrument 260 in FIG. 14). The surgical device obtained at step 504 may be a sterile surgical device, and may include a handle and a shaft extending from the handle along a shaft axis, wherein the handle comprises a grip, and wherein the grip extends along a grip axis oriented transverse to the shaft axis.

At step 506, the method further includes positioning the medical device into the thermoformed tray in an upright orientation. For example, in the upright orientation, the grip axis of the surgical device may be oriented transverse to the base of the tray, and the shaft axis of the surgical device is oriented transverse to the proximal sidewall and the distal sidewall and parallel to the longitudinal axis of the tray. In various aspects, the surgical device may be moved toward the base of the tray and into the tray's receptacle, for example, along the upright grip axis defined by the grip of the surgical device handle.

In various instances, thermoforming the sheet into the tray includes stretching the plurality of sidewalls to different heights to create different wall thicknesses along the length of the receptacle. For example, the deepest wells of the receptacle in the tray have the thinnest minimum sidewall thickness, which may facilitate deformation of the sidewalls by a clinician during use to grip the surgical device (e.g. the pistol grip of a surgical device handle) positioned in the receptacle.

In various instances, the method 500 may further include adding texture to an outer surface of the tray. For example, texture may be added to the lateral sidewalls of the tray handle to facilitate gripping thereof by the clinician.

The method 500 may further include loading accessories for the surgical device into the tray. For example, a coiled power cord may be loaded into the tray in an upright orientation and adjacent to the handle of the surgical device. The method 500 may further include sterilizing the surgical device and/or accessories therefor.

The method 500 may further include adding one or more retainers or inserts into the tray to support the surgical device and/or accessories therefor. The method 500 may further include adhering a peelable film to a rimmed perimeter of the tray to enclose the surgical device within the tray.

It is to be understood that the mention of one or more steps of a method does not preclude the presence of additional method steps or intervening method steps between those steps expressly identified. Similarly, some steps of a method may be performed in a different order than those described herein without departing from the scope of the disclosed technology.

Medical devices include surgical devices and surgical implants, for example. Surgical devices may include handheld surgical devices (e.g. handheld surgical instruments) and/or robotically-controlled surgical devices (e.g. robotic tools). Robotic surgical tools may include a proximal handle or housing that is attachable to a robot. In at least one such embodiment, the housing comprises rotatable inputs that are operably coupled with motor-driven outputs of the robotic surgical instrument system when the housing assembly is attached to the robot. Surgical devices may include surgical graspers, cutters, staplers, clip appliers, catheters, access devices, drug/gene therapy delivery devices, and energy delivery device using ultrasonic vibration, RF, laser, etc. The surgical device stored in the containers disclosed herein may be an endoscopic surgical device having an elongated shaft that is insertable through a trocar to a desired depth. The surgical device may be used to effect patient tissue, such as to manipulate, grasp, energize, clamp, staple, and/or suture tissue. Additionally or alternatively, the surgical device may be used to illuminate and/or image a surgical site, such as an endoscope, for example.

The terms “distal” and “proximal” are used throughout the following description and are meant to refer to positions and directions relative to a treating clinician. As such, “distal” or distally” refer to a position farther away or in a direction away from the clinician while “proximal” or “proximally” refer to a position nearer to or in a direction towards the clinician. As an example, the handle of a handheld surgical instrument may be proximal to the end effector of the handheld surgical instrument. The shaft of the handheld surgical instrument may extend distally from the handle of the handheld surgical instrument.

Surgical devices may be stored, transported, and used in a number of different orientations. Moreover, certain surgical devices can be repositioned by a clinician. For example, a surgical device can be articulated between a non-articulated configuration and an articulated position to rotate an end effector relative to the shaft and/or the handle.

Examples of the devices, systems, and methods disclosed herein are provided in the following numbered clauses. An aspect of the devices, systems, and methods may include any one, more than one, and any combination of the numbered clauses below:

Clause 1. A container, comprising: a tray comprising a base, a rim, and a plurality of sidewalls extending between the base and the rim, wherein the base and the plurality of sidewalls form a receptacle comprising a varying depth, and wherein the plurality of sidewalls comprises a plurality of handle sidewalls extending from the rim to the base at a peak depth along the varying depth of the receptacle; and a surgical device positioned in the receptacle, wherein the surgical device comprises a handle and a shaft extending from the handle along a shaft axis, wherein the handle comprises a grip extending along a grip axis oriented transverse to the shaft axis, and wherein the grip axis extends through the base of the tray to orient the handle in an upright orientation in the receptacle.

Clause 2. The container of Clause 1, wherein the tray defines a width and a length, wherein the shaft axis is oriented along the length of the tray, and wherein the peak depth is greater than the width and less than the length.

Clause 3. The container of Clause 1, wherein the handle further comprises a peak height, wherein the peak depth is greater than the peal height, and wherein handle at the peak height overlies the peak depth of the receptacle.

Clause 4. The container of Clause 2, wherein the grip comprises a pair of opposing lateral sides, and wherein the plurality of handle sidewalls are adjacent to the pair of opposing lateral sides.

Clause 5. The container of Clause 1, wherein the surgical device comprises a sterile surgical device.

Clause 6. The container of Clause 1, wherein the surgical device further comprises an end effector extending distally from the shaft, and wherein the base further comprises a crest positioned to support the shaft at a location intermediate the handle and the end effector.

Clause 7. The container of Clause 6, wherein the receptacle further comprises a distal cavity, and wherein the end effector is suspended in the distal cavity of the receptacle.

Clause 8. The container of Clause 1, wherein the surgical device further comprises a coiled cord positioned in the receptacle, wherein the coiled cord is oriented in an upright orientation substantially parallel to the grip axis.

Clause 9. A container, comprising: a tray comprising a base, a rim, and a plurality of sidewalls extending between the base and the rim, wherein the base and the plurality of sidewalls form a receptacle, and wherein the receptacle comprises: a first end; a second end opposite to the first end; and a varying depth comprising a peak depth intermediate the first end and the second end; wherein the plurality of sidewalls comprises a plurality of handle sidewalls extending to the base at the peak depth of the receptacle, wherein the plurality of handle sidewalls form a tray handle, and wherein the plurality of handle sidewalls are laterally spaced to accommodate a handle of a surgical device and sized to deflect inwardly toward opposing lateral sides of the handle of the surgical device when manually grasped.

Clause 10. The container of Clause 9, wherein the plurality of sidewalls further comprises a distal pair of sidewalls extending from the base to the rim, wherein the distal pair of sidewalls are distal to the plurality of handle sidewalls, and wherein a minimum wall thickness of the distal pair of sidewalls is greater than the minimum wall thickness of the handle sidewalls.

Clause 11. The container of Clause 10, wherein the plurality of handle sidewalls are more flexible than the distal pair of sidewalls.

Clause 12. The container of Clause 9, wherein the plurality of handle sidewalls comprises an outer surface comprising a textured grip.

Clause 13. The container of Clause 9, wherein the rim defines an opening into the receptacle, wherein the opening defines an opening surface area, wherein the receptacle defines a volume, and wherein the ratio of the volume to the opening surface area is greater than 1.5 inches.

Clause 14. The container of Clause 9, wherein the rim defines an opening into the receptacle, wherein the opening defines a perimeter length, wherein the receptacle defines a volume, and wherein the ratio of the volume to the perimeter length is greater than 3.0 inches

Clause 15. The container of Clause 14, further comprising a flexible film adhered to the rim along the perimeter length.

Clause 16. The container of Clause 15, further comprising the surgical device, wherein the surgical device is positioned in an upright orientation in the receptacle, and wherein the surgical device is encased within the receptacle between the tray and the flexible film.

Clause 17. The container of Clause 16, the tray further comprising a shoulder extending along at least a portion of the rim, the container further comprising a tray insert dimensioned to sit on the shoulder of the tray, and wherein the tray insert comprises a plurality of contours structured to complement an upper surface of the surgical device positioned in the upright orientation in the receptacle.

Clause 18. A set, comprising: a first surgical device packaging tray comprising a first base and a first plurality of sidewalls extending from the first base to form a first receptacle, wherein the first base comprises a first outward-facing surface, and wherein the first outward-facing surface comprises a first protrusion and a first indentation of the first base; and a second surgical device packaging tray comprising a second base and a second plurality of sidewalls extending from the second base to form a second receptacle, wherein the second base comprises a second outward-facing surface, and wherein the second outward-facing surface comprises a second protrusion and a second indentation of the first base; wherein the first outward-facing surface faces the second outward-facing surface, and wherein the first protrusion is configured to nest in the second indentation and the second protrusion is configured to nest in the first indentation.

Clause 19. The set of Clause 18, further comprising: a first surgical device positioned in the first surgical device packaging tray; and a second surgical device positioned in the second surgical device packaging tray.

Clause 20. The set of Clause 19, wherein the first surgical device comprises a handle, a shaft, and an end effector, wherein the first protrusion is dimensioned to receive the handle, and wherein the first indentation is structured to support the shaft at a location between the handle and the end effector.

Clause 21. The set of Clause 18, further comprising a carton dimensioned to receive the first surgical device packaging tray and the second surgical device packaging tray nested base-to-base in the carton.

Clause 22. A method, comprising: obtaining a sterile surgical device comprising a handle and a shaft extending from the handle along a shaft axis, wherein the handle comprises a grip, and wherein the grip extends along a grip axis oriented transverse to the shaft axis; obtaining a tray comprises a base and a plurality of sidewalls extending from the base to form a receptacle, wherein the plurality of sidewalls comprises a proximal sidewall and a distal sidewall; and positioning the sterile surgical device in the tray in an upright orientation, wherein, in the upright orientation, the grip axis is oriented transverse to the base and the shaft axis is oriented transverse to the proximal sidewall and the distal sidewall.

Clause 23. The method of Clause 22, further comprising thermoforming a sheet into the tray.

Clause 24. The method of Clause 23, wherein thermoforming the sheet into the tray further comprises stretching the plurality of sidewalls to different heights to create different wall thicknesses along the length of the receptacle.

Clause 25. The method of Clause 23, further comprising adding texture to an outer surface of the tray.

Clause 26. The method of Clause 22, wherein positioning the sterile surgical device in the tray in the upright orientation comprises moving the sterile surgical device toward the base of the tray along the grip axis, and wherein the grip axis comprises an upright axis.

The singular forms “a,” “an,” and “the” include plural referents unless the context clearly dictates otherwise. Furthermore, the use of “couple”, “coupled”, or similar phrases should not be construed as being limited to a certain number of components or a particular order of components unless the context clearly dictates otherwise.

As used herein, the terms “about” or “approximately” for any numerical values or ranges indicate a suitable dimensional tolerance (e.g. +20%) that allows the part or collection of components to function for its intended purpose as described herein.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material. None is admitted to be prior art.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1-26. (canceled)

16. A container, comprising:
a tray comprising a base, a rim, and a plurality of sidewalls extending between the base and the rim, wherein the base and the plurality of sidewalls form a receptacle comprising a varying depth, and wherein the plurality of sidewalls comprises a plurality of handle sidewalls extending from the rim to the base at a peak depth along the varying depth of the receptacle; and
a surgical device positioned in the receptacle, wherein the surgical device comprises a handle and a shaft extending from the handle along a shaft axis, wherein the handle comprises a grip extending along a grip axis oriented transverse to the shaft axis, and wherein the grip axis extends through the base of the tray to orient the handle in an upright orientation in the receptacle.

17. The container of claim 16, wherein the tray defines a width and a length, wherein the shaft axis is oriented along the length of the tray, and wherein the peak depth is greater than the width and less than the length.

18. The container of claim 16, wherein the handle further comprises a peak height, wherein the peak depth is greater than the peal height, and wherein handle at the peak height overlies the peak depth of the receptacle.

19. The container of claim 17, wherein the grip comprises a pair of opposing lateral sides, and wherein the plurality of handle sidewalls are adjacent to the pair of opposing lateral sides.

20. The container of claim 16, wherein the surgical device comprises a sterile surgical device.

21. The container of claim 16, The container of claim 16, wherein the surgical device further comprises an end effector extending distally from the shaft, and wherein the base further comprises a crest positioned to support the shaft at a location intermediate the handle and the end effector.

22. The container of claim 21, wherein the receptacle further comprises a distal cavity, and wherein the end effector is suspended in the distal cavity of the receptacle.

23. The container of claim 16, wherein the surgical device further comprises a coiled cord positioned in the receptacle, wherein the coiled cord is oriented in an upright orientation substantially parallel to the grip axis.

24. A container, comprising:
a tray comprising a base, a rim, and a plurality of sidewalls extending between the base and the rim, wherein the base and the plurality of sidewalls form a receptacle, and wherein the receptacle comprises:
a first end;
a second end opposite to the first end; and
a varying depth comprising a peak depth intermediate the first end and the second end,
wherein the plurality of sidewalls comprises a plurality of handle sidewalls extending to the base at the peak depth of the receptacle,
wherein the plurality of handle sidewalls form a tray handle, and
wherein the plurality of handle sidewalls are laterally spaced to accommodate a handle of a surgical device and sized to deflect inwardly toward opposing lateral sides of the handle of the surgical device when manually grasped.

25. The container of claim 24, wherein the plurality of sidewalls further comprises a distal pair of sidewalls extending from the base to the rim, wherein the distal pair of sidewalls are distal to the plurality of handle sidewalls, and wherein a minimum wall thickness of the distal pair of sidewalls is greater than the minimum wall thickness of the handle sidewalls.

26. The container of claim 25, wherein the plurality of handle sidewalls are more flexible than the distal pair of sidewalls.

27. container of claim 24, wherein the plurality of handle sidewalls comprises an outer surface comprising a textured grip.

28. The container of claim 24, wherein the rim defines an opening into the receptacle, wherein the opening defines an opening surface area, wherein the receptacle defines a volume, and wherein the ratio of the volume to the opening surface area is greater than 1.5 inches.

29. The container of claim 24, wherein the rim defines an opening into the receptacle, wherein the opening defines a perimeter length, wherein the receptacle defines a volume, and wherein the ratio of the volume to the perimeter length is greater than 3.0 inches.

30. The container of claim 29, further comprising a flexible film adhered to the rim along the perimeter length.

31. The container of claim 30, further comprising the surgical device, wherein the surgical device is positioned in an upright orientation in the receptacle, and wherein the surgical device is encased within the receptacle between the tray and the flexible film.

32. The container of claim 31, the tray further comprising a shoulder extending along at least a portion of the rim, the container further comprising a tray insert dimensioned to sit on the shoulder of the tray, and wherein the tray insert comprises a plurality of contours structured to complement an upper surface of the surgical device positioned in the upright orientation in the receptacle.

33. A set, comprising:
a first surgical device packaging tray comprising a first base and a first plurality of sidewalls extending from the first base to form a first receptacle, wherein the first base comprises a first outward-facing surface, and wherein the first outward-facing surface comprises a first protrusion and a first indentation of the first base; and
a second surgical device packaging tray comprising a second base and a second plurality of sidewalls extending from the second base to form a second receptacle, wherein the second base comprises a second outward-facing surface, and wherein the second outward-facing surface comprises a second protrusion and a second indentation of the first base,
wherein the first outward-facing surface faces the second outward-facing surface, and wherein the first protrusion is configured to nest in the second indentation and the second protrusion is configured to nest in the first indentation

34. The set of claim 33, further comprising:
a first surgical device positioned in the first surgical device packaging tray; and
a second surgical device positioned in the second surgical device packaging tray.

35. The set of claim 34, wherein the first surgical device comprises a handle, a shaft, and an end effector, wherein the first protrusion is dimensioned to receive the handle, and wherein the first indentation is structured to support the shaft at a location between the handle and the end effector.

36. The set of claim 33, further comprising a carton dimensioned to receive the first surgical device packaging tray and the second surgical device packaging tray nested base-to-base in the carton.

37. A method, comprising:
obtaining a sterile surgical device comprising a handle and a shaft extending from the handle along a shaft axis, wherein the handle comprises a grip, and wherein the grip extends along a grip axis oriented transverse to the shaft axis;
obtaining a tray comprises a base and a plurality of sidewalls extending from the base to form a receptacle, wherein the plurality of sidewalls comprises a proximal sidewall and a distal sidewall;
positioning the sterile surgical device in the tray in an upright orientation, wherein, in the upright orientation, the grip axis is oriented transverse to the base and the shaft axis is oriented transverse to the proximal sidewall and the distal sidewall.

38. The method of claim 22, further comprising thermoforming a sheet into the tray.

39. The method of claim 38, wherein thermoforming the sheet into the tray further comprises stretching the plurality of sidewalls to different heights to create different wall thicknesses along the length of the receptacle.

40. The method of claim 38, further comprising adding texture to an outer surface of the tray.

41. The method of claim 37, wherein positioning the sterile surgical device in the tray in the upright orientation comprises moving the sterile surgical device toward the base of the tray along the grip axis, and wherein the grip axis comprises an upright axis.
